(19) United States
(12) Patent Application Publication
(10) Pub. No.: US 2003/0162268 A1
(43) Pub. Date: Aug. 28, 2003

(54) DNA sequence encoding a retinoic acid regulated protein

(76) Inventors: Nancy Y. Ip, Hong Kong (HK); William M. W. Cheung, Sunshine City (HK)

Correspondence Address: DORITY & MANNING, P.A., POST OFFICE BOX 1449, GREENVILLE, SC, 29602-1449, US

(21) Appl. No.: 10/409,511

(22) Filed: Apr. 8, 2003

Related U.S. Application Data
(62) Division of application No. 09/354,359, filed on Jul. 14, 1999, now abandoned.

(51) Int. Cl. C12P 13/14; C07H 21/04; C12P 21/02; C12N 5/06; C07K 14/705

(52) U.S. Cl. 435/110; 435/69.1; 435/320.1; 435/325; 530/350; 536/23.5

(57) ABSTRACT

The present invention concerns a novel retinoic acid (RA) regulated gene whose expression product displays useful morphogenic/mitogenic properties.

### RELATED APPLICATIONS

This application is a Divisional of application Ser. No. 09,354,359 which is incorporated herein by reference.

The present invention concerns a novel retinoic acid (RA) regulated gene whose expression product displays useful morphogenic/mitogenic properties.

Retinoic acid induces the differentiation of many cell types, such as epithelial cells, mesenchyme cells, teratocarcinoma cells, leukaemia cells and immortalized cell lines such as embryonal carcinoma cells and neuroblastoma cells. RA is a morphogen which specifies axial patterning during embryonic development and which affects neurogenesis, and has been used as an effective therapeutic agent for the treatment of acute promyelocytic leukaemia.

The exact mode of action of retinoic acid is currently unknown, although it is known to be mediated by the nuclear retinoic acid receptors (RARs) (Chambon, P., 1996, FASEB J., 10: 940-959) and it is hypothesised that the diverse effects of RA result from the differential regulation of proteins such as transcription factors, enzymes and growth factor receptors.

Cheung, W. M. W. et al. (1997, J. Neurochem., 68: 1882-1888) have used RNA fingerprinting by arbitrarily primed PCR to identify a large number of genes that are differentially regulated during RA-induced neuronal differentiation. The present inventors have succeeded in isolating, purifying and cloning a novel gene which is down-regulated during RA-induced neuronal differentiation and whose resultant protein product possesses morphogenic/mitogenic properties.

According to the present invention there is provided a gene having the sequence of SEQ ID NO: 1. Also provided is an expression product encoded by the gene of the present invention, and in particular an expression product of the gene having the sequence of SEQ ID NO: 2. The present invention also extends to allelic mutants of said gene and gene expression product, and also to modified forms of said nucleic acid sequence which encode said expression product. For example, modifications may be made to the nucleic acid sequence such that it has a different sequence yet still codes for the same amino acid sequence.

Experiments (below) show that the expression product is important in maintaining the stem cell identity of the progenitor cells, as well as in the early differentiation of the progenitor cells. It is also important in embryogenesis and also appears to participate in the functioning of adult tissues, particularly brain, lung, liver and kidney. Expression of the gene product in lymphoid tissues shows a restrictive profile in the T-cell lineage of the immune system, particularly in the thymus and the bone marrow.

The gene of the present invention may also have applications in the treatment of Ushers disease, particularly type II Ushers disease, and thus the present invention extends to the use of the gene and its expression product in the manufacture of medicaments for treating Ushers disease, together with methods of treatment of Ushers disease.

Thus the gene of the present invention is useful both in treating and preventing diseases associated with its expression, with morphogeny and mitogeny, and with Ushers disease, particularly type II Ushers disease.

Thus the expression product according to the present invention may be a mitogen and/or a morphogen.

The expression product of the present invention may be usefully provided in the form of a recombinant construct, allowing its expression by chosen organisms under chosen conditions.

According to the present invention, there is also provided a DNA molecule, which may be in recombinant or isolated form, comprising a sequence encoding an expression product according to the present invention.

The coding sequence may be operatively linked to an expression control sequence sufficient to drive expression. Recombinant DNA in accordance with the invention may be in the form of a vector, for example a plasmid, cosmid or phage. A vector may include at least one selectable marker to enable selection of cells transfected (or transformed) with the vector. Such a marker or markers may enable selection of cells harbouring vectors incorporating heterologous DNA. The vector may contain appropriate start and stop signals. The vector may be an expression vector having regulatory sequences to drive expression. Vectors not having regulatory sequences may be used as cloning vectors (as may expression vectors).

Cloning vectors can be introduced into suitable hosts (for example E. coli) which facilitate their manipulation. According to another aspect of the invention, there is therefore provided a host cell transfected or transformed with DNA according to the present invention. Such host cells may be prokaryotic or eukaryotic. Expression hosts may be stably transformed. Unstable and cell-free expression systems may of course also be used.

Expression hosts may contain other exogenous DNA to facilitate the expression, assembly, secretion and other aspects of the biosynthesis of molecules of the invention.

The invention may be used with synthetic DNA sequences, cDNAs, full genomic sequences and “minigenes”, i.e. partial genomic sequences containing some, but not all, of the introns present in the full length gene.

DNA according to the present invention may be prepared by any convenient method involving coupling together successive nucleotides, and/or ligating oligo- and/or poly-nucleotides, including in vitro processes, as well as by the more usual recombinant DNA technology.

Also provided according to the present invention is a recombinant DNA construct comprising operatively linked in sequence in the 5′ to 3′ direction:

a) a promoter region that directs the transcription of a gene;

b) a DNA coding sequence encoding an RNA sequence encoding an expression product of the present invention; and

c) a 3′ non-translated region.

The DNA coding sequence may have the sequence of SEQ ID NO: 1.

Also provided is a cell transformed or transfected with a recombinant DNA construct of the present invention.

Also provided is a method of treating or preventing diseases associated with the expression of a gene of the present invention, comprising administering to a patient an expression product of the present invention.

As well as simply expressing the gene or administering the gene product in order to effect treatment of a patient, it may also be desirable to inhibit (i.e. antagonise) the gene product. This can be achieved in a multitude of ways, as will be readily apparent to one skilled in the art, and the teachings of U.S. Pat. No. 5,856,129 and references cited therein on how to produce and identify antagonists, inhibitors and potentiators of gene products are incorporated herein by refernce. In particular, the following teachings may be used: Harlow, E. and Lane, D., “Using Antibodies: A Laboratory Manual”, Cold Spring Harbor Laboratory Press, New York, 1998; Sambrook, J., Frisch, E. F., and Maniatis, T., “Molecular Cloning. A Laboratory Manual”, Cold Spring Harbor Laboratory, Cold Spring Harbor Press, New York, 1989; Ausubel, F. M. et al., 1989, Current Protocols in Molecular Biology, John Wiley & Sons, New York, N.Y.; Gee, J. E. et al., 1994, In: Huber, B. E. and Carr, B. I. Molecular and Immunologic Approaches, Futura Publishing Co., Mt. Kisco, N.Y.

The invention will be further apparent from the following description with reference to the figures, which shows by way of example only the cloning and study of the gene of the present invention.

Of the Figures:

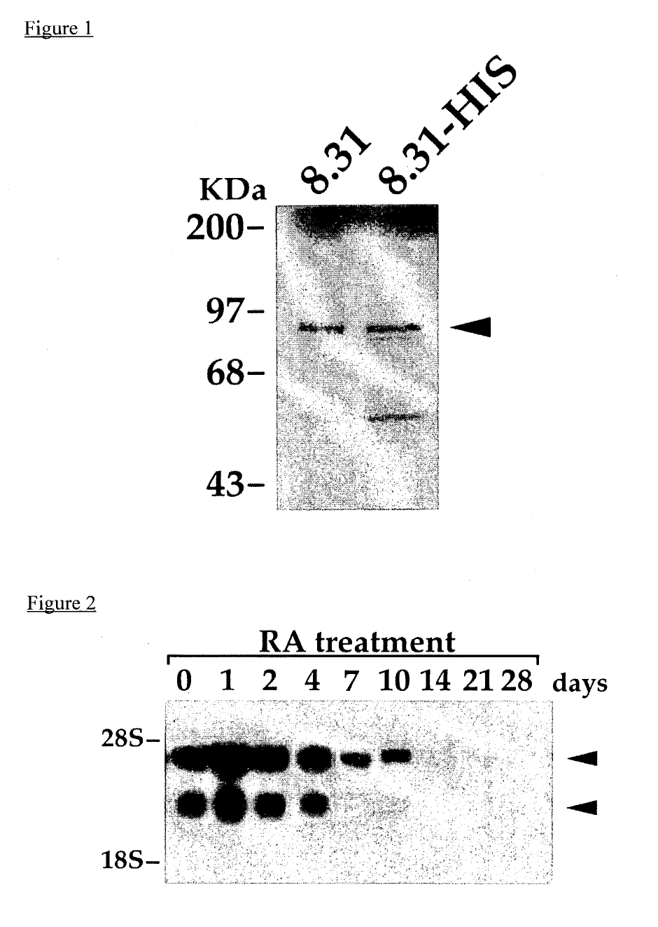

FIG. 1 shows coupled in vitro transcription and translation using rabbit reticulocyte extract, demonstrating that full length 8.31 cDNA encoded a ˜80 kDa protein. Histidine (His)-tagged 8.31 protein was constructed by cloning 6 His to the C-terminus of 8.31. Coupled in vitro transcription and translation was performed in the absence of radioactive label. The translated proteins were separated by SDS PAGE, transferred to nitrocellulose membrane and blotted with monoclonal antibody against the 6× His tail.

FIG. 2 shows Northern blot analysis of 8.31 expression in RA-treated NT2 cells. Total RNA (10 μg) prepared from NT2 cells treated with all-trans RA for 0 to 28 days, separated by denaturing gel electrophoresis, and transferred to nylon membrane. Hybridization was performed using the full length 8.31 cDNA as probe. Ribosomal RNA bands are as shown on the left.

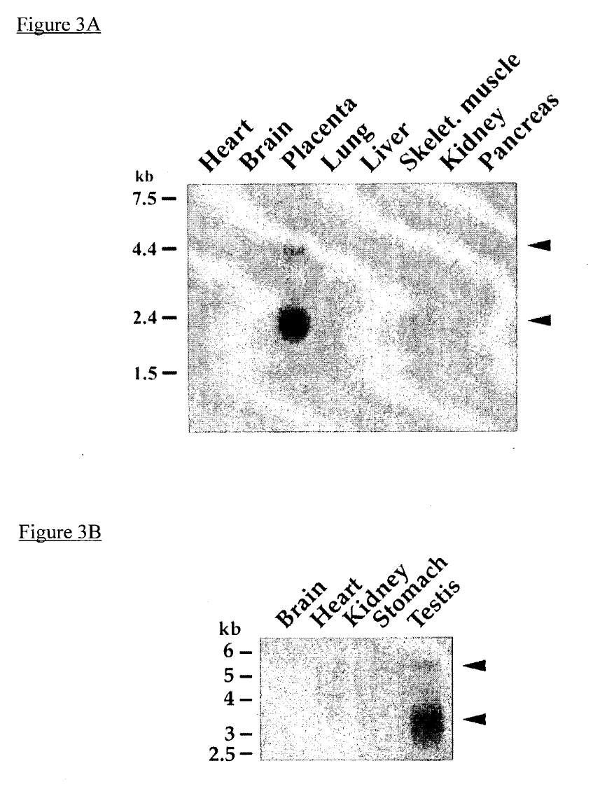

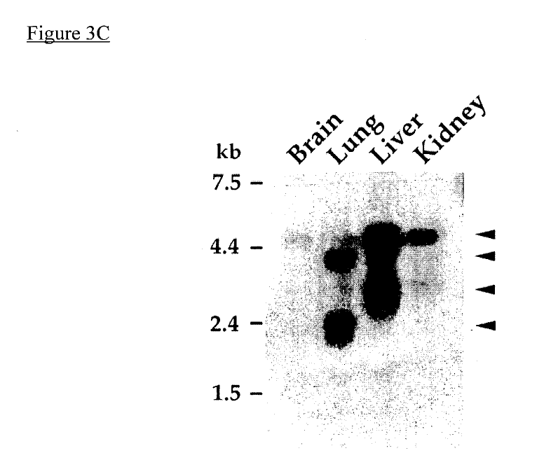

FIG. 3 shows the expression profile of 8.31 in human tissues. Multiple Tissue Northern blots (Clontech) were hybridized using full length 8.31 cDNA as probe. Results of the hybridization using adult tissues (FIGS. 3A and 3B) and fetal tissues are shown (FIG. 3C). RNA size markers are indicated on the left.

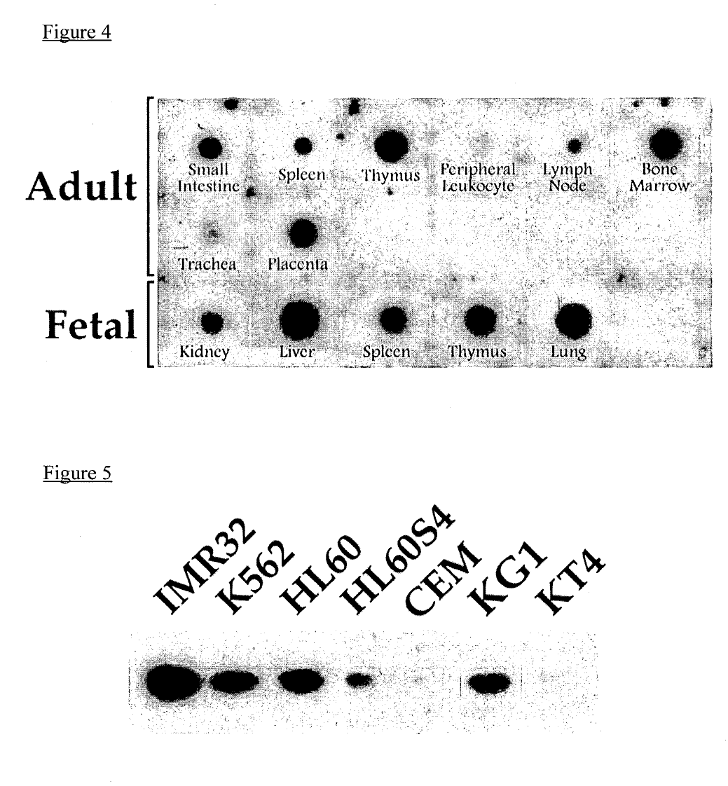

FIG. 4 shows a dot blot analysis of 8.31 expression. Messenger RNA (2 μg) was used in the dot blot to examine the expression of 8.31 in various tissues of hematopoietic origin as well as fetal tissues. Results of the hybridization using full length 8.31 cDNA as probe are shown. Adult cells (top and middle rows) are (left to right, top to bottom) small intestine, spleen, thymus, peripheral leukocyte, lymph node, bone marrow, trachea and placenta. Fetal cells (bottom row) are (left to right) kidney, liver, spleen thymus and lung.

FIG. 5 shows RT-PCR analysis of the 8.31 expression in human cell lines. Total RNA (2 μg) obtained from neuronal precursor cell lines IMR32, and leukaemia cells was reverse transcribed and amplified by specific primers for 8.31. KT4 represents treatment of KG1 cells with all-trans RA for 4 days. Hybridization was performed to confirm the identity of the amplified products.

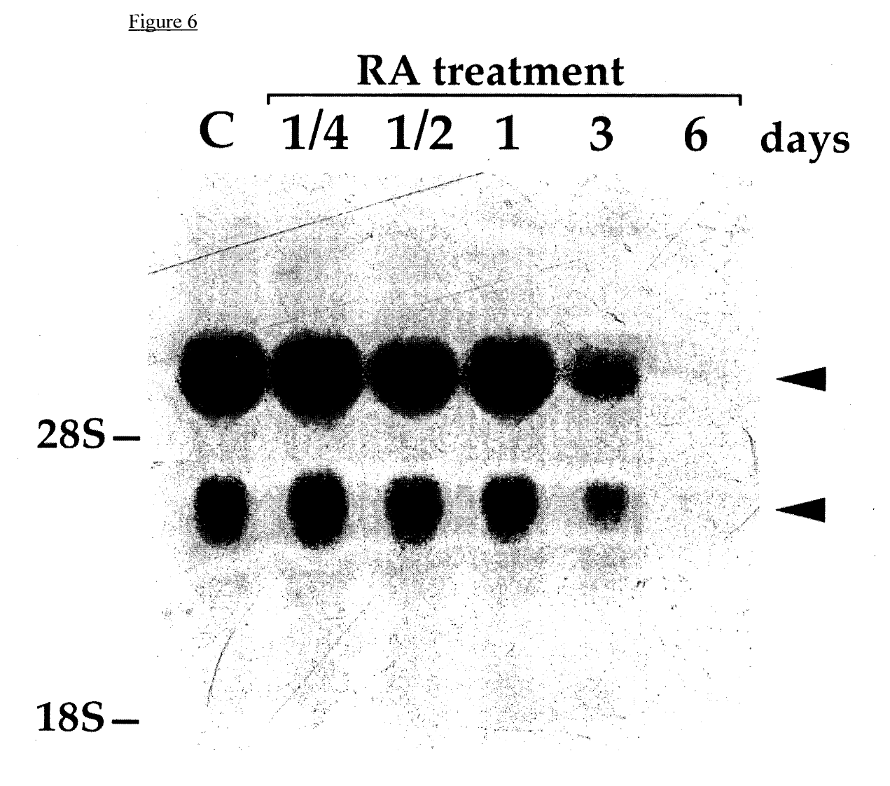

FIG. 6 shows expression of 8.31 in RA-treated HL-60 cells. Total RNA (15 μg) from HL-60 cells treated with 0 to 6 days was used for Northern blot analysis using full length 8.31 cDNA probe. Ribosomal RNA bands are indicated on the left.

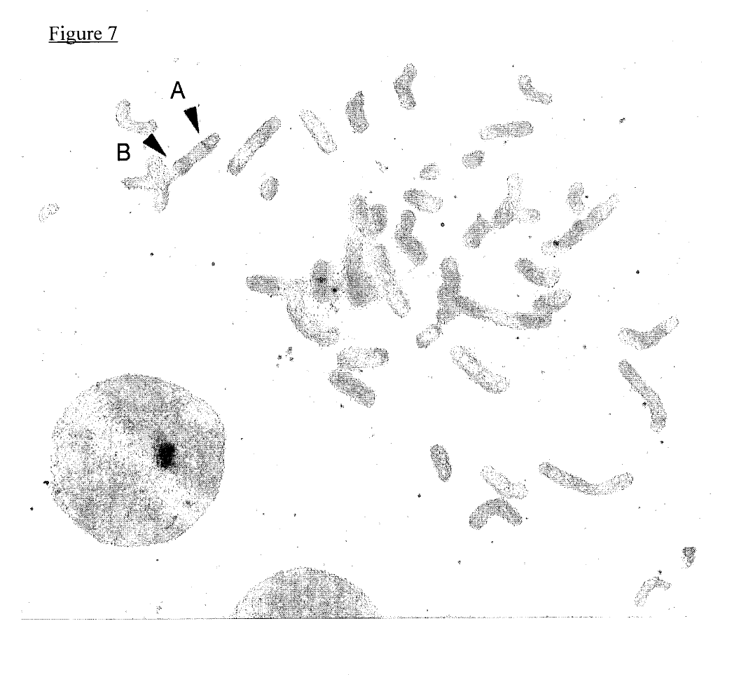

FIG. 7 shows chromosomal localization of the gene 8.31 by FISH. Gene 8.31 was labelled and is shown marked “A”, and the specific marker for the heterochromatin of chromosome 1 was labelled and so is shown marked “B”.

### EXPERIMENTAL

The gene of the present invention (also referred to as clone 8.31) was cloned and expressed, its in vitro transcription and translation assayed and its chromosomal location determined. The expression profile of 8.31 in a range of cell types and under a range of conditions has allowed a role for it to be determined.

### Materials and Methods

Experimental methods referred to and used are standard laboratory techniques. Where specific methods are not described or referenced, full descriptions and protocols are well known in the art and available in laboratory manuals such as Harlow, E. and Lane, D., “Using Antibodies: A Laboratory Manual”, Cold Spring Harbor Laboratory Press, New York, 1998; Sambrook, J., Frisch, E. F., and Maniatis, T., “Molecular Cloning. A Laboratory Manual”, Cold Spring Harbor Laboratory, Cold Spring Harbor Press, New York, 1989; PCR (Volume 1): A practical approach. Eds. M. J. McPherson, P. Quirke and G. R. Taylor. Oxford University Press, 1991; and Torres, R. M. and Kühn, R., “Laboratory Protocols for Conditional Gene Therapy”, Oxford University Press, 1997, ISBN 019963677-X.

Cloning of full length cDNA of 8.31

Full length cDNA of 8.31 was obtained by screening an expression cDNA library prepared from undifferentiated NT2 cells (STRATAGENE) using the partial 8.31 cDNA fragment (SEQ ID NO: 3) as probe. Radioactive cDNA probes were prepared using the Megaprime DNA labelling system (AMERSHAM). Single phages were obtained and transformed into XLOLR bacterial cells (STRATAGENE) and the cDNA fragment cloned into pBK-CMV mammalian expression vector by in vivo excision.

Cell culture

NT2 cells were cultured as previously described (Cheung et al., 1996, NeuroReport, 6: 1204-1208). Cells were maintained in Opti-MEM I reduced-serum medium (GIBCO) supplemented with 5% fetal bovine serum (FBS, GIBCO). NT2 cells were differentiated with 5 μM all-trans RA (SIGMA) in Dulbecco's modified Eagle's medium (DMEM; high glucose formulation) supplemented with 10% FBS. Leukaemia cell lines were cultured as previously described (Xie et al., 1997, NeuroReport, 8: 1067-1070).

8.31 cDNA probe

The partial cDNA sequence of 8.31 was obtained using RNA fingerprinting by arbitrarily primed PCR (RAP-PCR, Welsh, J. et al., 1992, Nucleic Acids Res., 20: 4965-4970). Total RNA was obtained from NT2 cells treated for various durations with all-trans RA (10 M). The differentially-regulated cDNA fragments were cloned into pCRscript SK+ for DNA sequencing. The cDNA probe (SEQ ID NO: 3) was then used to screen an undifferentiated human NT2 cell cDNA library for the full length 8.31 cDNA.

RNA preparation, RT-PCR and Northern blot analysis

Total RNA was prepared using Trizol reagent (GIBCO) or as previously described (Xie et al., 1997, NeuroReport, 8: 1067-1070). Equal amounts of total RNA from different cell lines were used for Northern blot analysis, while 2 μg total RNA was used for reverse transcription using Superscript II reverse transcritptase (GIBCO). One tenth of the reaction was amplified using Taq DNA polymerase (GIBCO). Gene expression was confirmed by using different numbers of PCR cycles and hybridization using 8.31 specific cDNA probes.

Coupled in vitro-transcription and translation

Two micrograms of plasmids were used for each coupled in vitro transcription/translation reaction using the TNT coupled reticulocyte lysate system (PROMEGA).

Chromosomal localization of 8.31 by FISH (fluorescent in situ hybridisation)

Genomic DNA encoding 8.31 was labelled with digoxigenin (DIG) dUTP by nick translation and was hybridized to normal metaphase chromosomes derived from PHA phytohemaglutinin stimulated peripheral blood lymphocytes. After incubation with fluorescein-conjugated anti-DIG antibodies, the cells were counterstained with DAPI (4,6-diamidino-2-phenylindole), a fluorescent DNA groove-binding probe.

### RESULTS

Cloning of full length coding sequence of 8.31

The cDNA encoding the full length 8.31 was obtained from a cDNA library prepared from undifferentiated NT2 cells using hybridization screening. Double stranded sequencing by T7 DNA polymerase revealed that the cDNA (˜2831 bp) is novel in its gene identity (FIG. 1). The coding sequence can be translated into a protein of 730 amino acid residues. Coupled in vitro transcription and translation was performed to demonstrate that the cloned cDNA can be translated into a protein with molecular weight of ˜80 kDa (FIG. 1).

Transcript expression of 8.31

The full length 8.31 was then used as a probe to examine its expression when the NT2 cells were treated with RA for 0 to 28 days (FIG. 2). Two transcripts were obtained (˜4.5 kb and ˜3.5 kb). The expression of 8.31 was slightly induced after 1 day of RA treatment. At day 2, the expression decreased to its basal level and then continue to decrease along the course of RA treatment. Its expression was almost halted at day 28.

To obtain clues on the potential functions of 8.31, we have examined the expression profile of 8.31 in both adult and fetal human tissues. Among the adult tissues examined, which include heart, brain, placenta, lung, liver, skeletal muscle, kidney, pancreas, stomach, and testis, prominent expression of 8.31 was observes in placenta and testis. Skeletal muscles expressed low levels of 8.31 (FIG. 3, panels A and B).

The expression of 8.31 was observed in all the human fetal tissues examined, which included brain, lung, liver and kidney. An extra transcript (˜5.5 kb) was observed in all fetal tissues and a small transcript (˜2.4 kb) was observed only in the messenger RNA prepared from the fetal lung (FIG. 3, panel C). The high expression of 8.31 in the fetal tissues examined was not observed in the corresponding adult tissues.

Dot blot analysis was performed to examine the expression of 8.31 in hematopoietic tissues. Expression of 8.31 was detected in all hematopoietic tissues examined; however, 8.31 was predominantly expressed in the thymus and the bone marrow. Lower transcript expression of 8.31 was detected in the spleen and lymph node. Only a barely detectable level of its expression was observed in the peripheral leukocytes (FIG. 4).

Expression of 8.31 in different human cell lines

Owing to the high expression of 8.31 detected in the hematopoietic tissues, we have examined its expression in several leukaemia cell lines to obtain clues on its roles in hematopoietic systems. RT PCR analysis was performed using total RNA prepared from K562, KG1, HL-60, HL-60S4, and CEM, cell lines each corresponding to a different type of leukaemia (FIG. 5). Transcript expression of 8.31 was observed in all hematopoietic cell lines tested. Its expression was also observed in a human neuroblastoma cell line, IMR32 cells.

Expression of 8.31 was down-regulated in RA-treated HL-60 and KG1 cells

HL-60 cells were differentiated with 1 μM all-trans RA and the expression of 8.31 was examined by Northern blot analysis. Two transcripts (˜5.5 kb and ˜3.5 kb) were detected in undifferentiated HL-60 cells (FIG. 6). When HL-60 cells were treated with RA for 3 days, its expression was significantly down-regulated (FIG. 6). At day 6 of RA treatment, the expression of 8.31 was diminished.

The expression of 8.31 was also down-regulated when KG1 cells were treated with 1 μM all-trans RA, as demonstrated by the RT-PCR analysis (FIG. 5).

Chromosomal localization of clone 8.31 by Fluorescence In Situ Hybridization

DNA from a genomic clone of 8.31 was labelled with digoxigenin dUTP by nick translation. Labelled probe was combined with sheared human DNA and hybridized to normal metaphase chromosomes derived from PHA stimulated peripheral blood lymphocytes. The initial experiment resulted in specific labelling of the long arm of a group A chromosome which was believed to be chromosome 1 on the basis of size, morphology, and banding pattern. A second experiment was conducted in which a biotin-labelled probe specific for the heterochromactic region of chromosome 1 was co-hybridized with the genomic clone of 8.31. This experiment resulted in a specific labelling of the heterochromatin in red (marked “B” in FIG. 7) and the long arm in green (marked “A” in FIG. 7) of the chromosome 1. Measurements of 10 specifically labelled chromosomes 1 demonstrated that the genomic clone of 8.31 is located at a position which is 62% of the distance from the heterochromatic-euchromatic boundary to the telomere of chromosome arm 1q, an area which corresponds to band 1q32.1-32.2 (FIG. 7). A total of 80 metaphase cells were analyzed with 76 exhibiting specific labelling.

Type II Ushers syndrome (classical retinitis pigmentosa combined with congenital pedial deafness, and normal vestibular function) has been mapped to the chromosomal region containing the gene of the present invention (Kimberling et al., 1990, Genomics, 2: 245-249); Lewis et al., 1990, Genomics, 2: 250-256) and it appears that the gene of the present invention, together with its expression product, may be useful in the treatment of Ushers syndrome. For example, the lack of function resulting from mutations in the diseased gene may be complemented by the gene and/or expression products of the present invention.

### DISCUSSION

Functional roles of 8.31

The expression profile observed for 8.31 suggests a potential role in tissues of hematopoietic origin. Recently, placental blood has been used as a rich source of hematopoietic stem cells for transplantation. Taken together with the high expression of 8.31 in the testis and the undifferentiated NT2 cells, the expression of 8.31 in placenta revealed a strong association of the gene to the identity of the stem cells. Hence it appears that the gene product of 8.31 is important in maintaining the stem cell identity of the progenitor cells, as well as in the early differentiation of the progenitor cells.

The expression of 8.31 is also strongly associated with the early embryonic development. This is exemplified by the high expression of 8.31 in fetal tissues such as brain, lung, liver and kidney, but not in same adult tissues. Together with its restrictive expression pattern in the adult tissues, it appears that the gene product of 8.31 is not only important in the embryogenesis, but is also participates in the functioning of these adult tissues. Different 8.31 isoforms exist, the expression of which can be regulated during the development (FIG. 3).

The predominant expession of 8.31 in the thymus and the bone marrow, but low expression in other lymphoid tissues revealed its restrictive functions in the T-cell lineage of the immune system.

Involvement of 8.31 in the differentiation of cancer cells

Northern blot analysis demonstrated the down-regulation of 8.31 expression with the treatment of all-trans RA. HL-60 is an acute promyelocytic leukaemia cell line. The growth rate was sharply decreased by treatment with RA. It appears that the expression of 8.31 is strongly associated with the differentiation of other cancer cell lines, including the embryonal carcinoma cells and the neuroblastoma cells. Hence 8.31 may serve as a diagnostic marker for different cancer types.

8.31 serves as a candidate gene for genetic diseases

The gene encoding 8.31 was localized to the chromosome 1q32.1 32.2 Chromosome 1q 32 locus has been mapped to several genetic diseases including the complement system malfunctioning, as well as the Usher disease, which is related to hearing. Moreover the Alzheimer's disease is also mapped to the region 1q32, although the exact position remains to be elucidated.

1. A gene having the sequence of SEQ ID NO: 1.

2. An expression product encoded by a gene according to claim 1.

3. An expression product according to claim 2, having the sequence of SEQ ID NO: 2.

4. An expression product according to claim 3, wherein said expression product is a mitogen.

5. An expression product according to claim 3, wherein said, expression product is a morphogen.

6. An expression product according to claim 2, wherein said expression product comprises a protein.

7. An expression product according to claim 2, wherein said expression product affects the identity of stem cell progenitor cells.

8. An expression product according to claim 2 which affects embryonic development.

9. An expression product according to claim 8, which affects the development of embryonic brain, lung, liver and kidney tissues.

10. An expression product according to claim 2, which affects cancer cell differentiation.

11. An expression product according to claim 2, for use in the treatment or prevention of Ushers Disease.

12. A recombinant DNA construct comprising operatively linked in sequence in the 5′ to 3′ direction:
a) a promoter region that directs the transcription of a gene;
b) a DNA coding sequence encoding an RNA sequence encoding an expression product according to claim 2; and
c) a 3′ non-translated region.

13. A recombinant DNA construct according to claim 12, the DNA coding sequence having the sequence of SEQ ID NO: 1.

14. A cell transformed or transfected with a recombinant DNA construct according to claim 12.

15. A method of treating or preventing diseases associated with the expression of a gene comprising administering to a patient an expression product according to claim 2.

16. A chromosome marker for band q 32.1-32.2 having the sequence of SEQ ID NO:2.


SEQUENCE LISTING (3 sequences)

SEQ ID NO: 1 - DNA, 2831 bp, Homo sapiens
  CDS (124)..(2313)
ggcacgagcgggagttggaggcgataacgatttgtgttgtgagaggcgcaacgtgcgatttctgctgaacttggaggcatttctacgacttttctctcagctgaggcttttcctccgaccctgatgctcttcaattcggtgctccgccagccccagcttggcgtcctgagaaatggatggtcttcacaataccctcttcaatcccttctgactggttatcagtgcagtggtaatgatgaacacacttcttatggagaaacaggagtcccagttcctccttttggatgtaccttctcttctgctcccaatatggaacatgtactagcagttgccaatgaagaaggctttgttcgattgtataacacagaatcacaaagtttcagaaagaagtgcttcaaagaatggatggctcactggaatgccgtctttgacctggcctgggttcctggtgaacttaaacttgttacagcagcaggtgatcaaacagccaaattttgggacgtaaaagctggtgagctgattggaacatgcaaaggtcatcaatgcagcctcaagtcagttgccttttctaagtttgagaaagctgtattctgtacgggtggaagagatggcaacattatggtctgggataccaggtgcaacaaaaaagatgggttttataggcaagtgaatcaaatcagtggagctcacaatacctcagacaagcaaaccccttcaaaacccaagaagaaacagaattcaaaaggacttgctccttctgtggatttccagcaaagtgttactgtggtcctctttcaagacgagaataccttagtctcagcaggagctgtggatgggataatcaaagtatgggatttacgtaagaattatactgcttatcgacaagaacccatagcatccaagtctttcctgtacccaggtagcagcactcgaaaacttggatattcaagtctgattttggattccactggctctactttatttgctaattgcacagacgataacatctacatgtttaatatgactgggttgaagacttctccagtggctattttcaatggacaccagaactctaccttttatgtaaaatccagccttagtccagatgaccagtttttagtcagtggctcaagtgatgaagctgcctacatatggaaggtctccacaccctggcaacctcctactgtgctcctgggtcattctcaagaggtcacgtctgtgtgctggtgtccatctgacttcacaaagattgctacctgttctgatgacaatacactaaaaatctggcgcttgaatagaggcttagaggagaaaccaggaggtgataaactttccacggtgggttgggcctctcagaagaaaaaagagtcaagacctggcctagtaacagtaacgagtagccagagtactcctgccaaagcccccagggtaaagtgcaatccatccaattcttccccgtcatccgcagcttgtgccccaagctgtgctggagacctccctcttccttcaaatactcctacgttctctattaaaacctctcctgccaaggcccggtctcccatcaacagaagaggctctgtctcctccgtctctcccaagccaccttcatctttcaagatgtcgattagaaactgggtgacccgaacaccttcctcatcaccacccatcactccacctgcttcggagaccaagatcatgtctccgagaaaagcccttattcctgtgagccagaagtcatcccaagcagaggcttgctctgagtctagaaatagagtaaagaggaggctagactcaagctgtctggagagtgtgaaacaaaagtgtgtgaagagttgtaactgtgtgactgagcttgatggccaagttgaaaatcttcatttggatctgtgctgccttgctggtaaccaggaagaccttagtaaggactctctaggtcctaccaaatcaagcaaaattgaaggagctggtaccagtatctcagagcctccgtctcctatcagtccgtatgcttcagaaagctgtggaacgctacctcttcctttgagaccttgtggagaagggtctgaaatggtaggcaaagagaatagttccccagagaataaaaactggttgttggccatggcagccaaacggaaggctgagaatccatctccacgaagtccgtcatcccagacacccaattccaggagacagagcggaaagacattgccaagcccggtcaccatcacgcccagctccatgaggaaaatctgcacatacttccatagaaagtcccaggaggacttctgtggtcctgaacactcaacagaattatagattctaatctgagtgagttactgagctttggtccactaaaacaagctgagctttggtccactaaaacaagatgaaaaatacaagagtgactctataactctggtctttaagaaagctgccttttcatttttagacaaaatcttttcaacgctgaaatgtacctaatctggttctactaccataatgtatatgcagcttcccgaggatgaatgctgtgtttaaatttcataaagtaaatttgtcactctagcattttgaatgaatagtcttcactttttaaattattcatcttctctataataatgacatcccagttcatggaggcaaaaaacaagtttcttgttatcctgaaactttctatgctcagtggaaagtatctgccagccacagcatgaggcctgtgaaggctgactgagaaatcctctgctgaagacccctggttctgttctgcctccaacatgtataattttatttgaaatacataatcttttcactatgaaaaaaaaaaaaaaaaa

SEQ ID NO: 2 - protein, 730 aa, Homo sapiens
MLFNSVLRQPQLGVLRNGWSSQYPLQSLLTGYQCSGNDEHTSYGETGVPVPPFGCTFSSAPNMEHVLAVANEEGFVRLYNTESQSFRKKCFKEWMAHWNAVFDLAWVPGELKLVTAAGDQTAKFWDVKAGELIGTCKGHQCSLKSVAFSKFEKAVFCTGGRDGNIMVWDTRCNKKDGFYRQVNQISGAHNTSDKQTPSKPKKKQNSKGLAPSVDFQQSVTVVLFQDENTLVSAGAVDGIIKVWDLRKNYTAYRQEPIASKSFLYPGSSTRKLGYSSLILDSTGSTLFANCTDDNIYMFNMTGLKTSPVAIFNGHQNSTFYVKSSLSPDDQFLVSGSSDEAAYIWKVSTPWQPPTVLLGHSQEVTSVCWCPSDFTKIATCSDDNTLKIWRLNRGLEEKPGGDKLSTVGWASQKKKESRPGLVTVTSSQSTPAKAPRVKCNPSNSSPSSAACAPSCAGDLPLPSNTPTFSIKTSPAKARSPINRRGSVSSVSPKPPSSFKMSIRNWVTRTPSSSPPITPPASETKIMSPRKALIPVSQKSSQAEACSESRNRVKRRLDSSCLESVKQKCVKSCNCVTELDGQVENLHLDLCCLAGNQEDLSKDSLGPTKSSKIEGAGTSISEPPSPISPYASESCGTLPLPLRPCGEGSEMVGKENSSPENKNWLLAMAAKRKAENPSPRSPSSQTPNSRRQSGKTLPSPVTITPSSMRKICTYFHRKSQEDFCGPEHSTEL

SEQ ID NO: 3 - DNA, 83 bp, Homo sapiens
tttgagaaagctgtattctgtacgggtggaagagatggcaacattatggtctgggataccaggtgcaacaaaaaagatgggtt